SEQUENCE LISTING (22 sequences)

SEQ ID NO: 1 - protein, 120 aa, synthetic construct
QVQLQQSGAELVRPGTSVKVSCKASGYAFSNYFIEWVKQRPGQGLEWIGLNIPGSGGSNYAEKFKGKATLTADKSSSTAYMQLSSLTSEDSAVYFCARRRLPPDWYFDVWGTGTTVTVSS

SEQ ID NO: 2 - protein, 120 aa, synthetic construct
QVQLVQSGAELKKPGSSVKVSCKASGYAFSNYFIEWVKQPPGKGLEWIGLNIPGSGGSNYAEKFKGRATITADKSTSTAYMELSSLTSEDSAVYFCARRRLPPDWYFDVWGQGTTVTVSS

SEQ ID NO: 3 - protein, 120 aa, synthetic construct
QVQLVQSGAEVKKPGSSVKVSCKASGYAFSNYFIEWVKQPPGKGLEWIGLNIPGSGGSNYAEKFKGRATITADKSTSTAYMELSSLRSEDSAVYFCARRRLPPDWYFDVWGQGTTVTVSS

SEQ ID NO: 4 - protein, 120 aa, synthetic construct
QVQLVQSGAEVKKPGSSVKVSCKASGYAFSNYFIEWVKQPPGKGLEWIGLNIPGSGGSNYAEKFKGRVTITADKSTSTAYMELSSLRSEDSAVYFCARRRLPPDWYFDVWGQGTTVTVSS

SEQ ID NO: 5 - protein, 120 aa, synthetic construct
QVQLVQSGAEVKKPGSSVKVSCKASGYAFSNYFIEWVKQPPGKGLEWIGLNIPGSGGSNYAEKFKGRVTITADKSTSTAYMELSSLRSEDTAVYYCARRRLPPDWYFDVWGQGTTVTVSS

SEQ ID NO: 6 - protein, 120 aa, synthetic construct
QVQLVQSGAEVKKPGSSVKVSCKASGYSFSNYFIEWVKQPPGKGLEWIGLNIPGSGGSNYAEKFKGRVTITADKSTSTAYMELSSLRSEDTAVYYCARRRLPPDWYFDVWGQGTTVTVSS

SEQ ID NO: 7 - protein, 120 aa, synthetic construct
QVQLVQSGAELKKPGSSVKVSCKASGYAFSNYFIEWVKQPPGKGLEWIGLNIPGSGGSNYAEKFKGRATITADKSTSTAYMELSSLRSEDTAVYYCARRRLPPDWYFDVWGQGTTVTVSS

SEQ ID NO: 8 - protein, 120 aa, synthetic construct
QVQLVQSGAEVKKPGSSVKVSCKASGYAFSNYFIEWVKQPPGKGLEWIGLNIPGSGGSNYAEKFKGRATITADKSTSTAYMELSSLRSEDTAVYYCARRRLPPDWYFDVWGQGTTVTVSS

SEQ ID NO: 9 - protein, 112 aa, synthetic construct
DVVMTQTPLSLPVSLGDQASISCRSSQSLVHSDGNTYLHWYLQKPGQSPKLLIYKVSNRFSGVPDRFSGSGSGTDFTLKISRVEAEDLGVYFCSQSTHVPWTFGGGTKLEIK

SEQ ID NO: 10 - protein, 112 aa, synthetic construct
DVVMTQSPLSLPVTLGQPASISCRSSQSLVHSDGNTYLHWYQQKPGQPPKLLIYKVSNRFSGVPDRFSGSGSGTDFTLKISRVEAEDVGVYFCSQSTHVPWTFGGGTKVEIK

SEQ ID NO: 11 - protein, 112 aa, synthetic construct
DVVMTQSPLSLPVTLGQPASISCRSSQSLVHSDGNTYLHWYQQKPGQPPKLLIYKVSNRFSGVPDRFSGSGSGTDFTLKISRVEAEDVGVYYCSQSTHVPWTFGGGTKVEIK

SEQ ID NO: 12 - protein, 112 aa, synthetic construct
DVVMTQSPLSLPVTLGQPASISCRSSQSLVHSGGNTYLHWYQQKPGQPPKLLIYKVSNRFSGVPDRFSGSGSGTDFTLKISRVEAEDVGVYFCSQSTHVPWTFGGGTKVEIK

SEQ ID NO: 13 - protein, 10 aa, synthetic construct
GYAFSNYFIE

SEQ ID NO: 14 - protein, 10 aa, synthetic construct
GYSFSNYFIE

SEQ ID NO: 15 - protein, 17 aa, synthetic construct
LNIPGSGGSNYAEKFKG

SEQ ID NO: 16 - protein, 11 aa, synthetic construct
RRLPPDWYFDV

SEQ ID NO: 17 - protein, 16 aa, synthetic construct
RSSQSLVHSGGNTYLH

SEQ ID NO: 18 - protein, 7 aa, synthetic construct
KVSNRFS

SEQ ID NO: 19 - protein, 9 aa, synthetic construct
SQSTHVPWT

SEQ ID NO: 20 - protein, 16 aa, synthetic construct
RSSQSLVHSDGNTYLH

SEQ ID NO: 21 - protein, 120 aa, synthetic construct; identical to SEQ ID NO: 1

SEQ ID NO: 22 - protein, 112 aa, synthetic construct; identical to SEQ ID NO: 9

(19) United States
(12) Patent Application Publication
(10) Pub. No.: US 2025/0179187 A1
(43) Pub. Date: Jun. 5, 2025

(54) ANTI PD-L2 ANTIBODY

(71) Applicants: REJUVERON SENESCENCE THERAPEUTICS AG, Schlieren (CH); REJUVERSEN EUROPE, S.L., Barcelona (ES)

(72) Inventors: Timothy CASH, Barcelona (ES); Marc RAMIS CASTELLTORT, Valldoreix (ES); Manuel SERRANO MARUGAN, Tres Cantos (ES); Joaquin ARRIBAS-LOPEZ, Barcelona (ES); Marta LALINDE-GUTIERREZ, Barcelona (ES)

(73) Assignees: REJUVERON SENESCENCE THERAPEUTICS AG, Schlieren (CH); REJUVERSEN EUROPE, S.L., Barcelona (ES)

(21) Appl. No.: 18/843,760

(22) PCT Filed: Mar. 3, 2023
(86) PCT No.: PCT/EP2023/055506

§ 371 Date: Sep. 4, 2024

(30) Foreign Application Priority Data
Mar. 4, 2022 (EP) 22382203.2

(51) Int. Cl. C07K 16/28; A61K 31/337; A61K 31/555; A61K 31/704; A61K 39/00; A61P 35/00

(52) U.S. Cl. CPC C07K 16/2827; A61K 31/337; A61K 31/555; A61K 31/704; A61P 35/00; A61K 2039/505; C07K 2317/24; C07K 2317/33; C07K 2317/565; C07K 2317/76; C07K 2317/92

(57) ABSTRACT

An isolated monoclonal antibody that specifically binds to programmed cell death ligand 2 (PD-L2) and blocks activity against PD-L2/hPD1 interaction, wherein the antibody includes six CDR sequences in the heavy chain variable region and in the light chain variable region, and includes as heavy chain variable region CDRs a CDRH1 region of SEQ ID NO: 13 (GYAFSNYFIE) or SEQ ID NO: 14 (GYSFSNYFIE); a CDRH2 region of SEQ ID NO: 15 (LNIPGSGGSNYAEKFKG); and a CDRH3 region of SEQ ID NO: 16 (RRLPPDWYFDV); and as light chain region CDRs a CDRL1 region of SEQ ID NO: 17 (RSSQSLVHSGGNTYLH) or SEQ ID NO: 20 (RSSQSLVHSDGNTYLH); a CDRL2 region of SEQ ID NO: 18 (KVSNRFS); and a CDRL3 region of SEQ ID NO: 19 (SQSTHVPWT).

The present invention relates to an isolated monoclonal antibody that binds to programmed cell death ligand 2 (PD-L2) and to its use as a medicament.

Senescent cells were found to accumulate in tissues and organs during the aging process at close proximity of age-related pathologies, where they play a critical role in the development and progression of age-related diseases and disorders. Cellular senescence is a complex stress response whereby cells irreversibly lose the capacity to proliferate, accompanied by numerous changes in gene expression. Many potentially oncogenic insults induce a senescence response, which is recognized as a potent tumor suppressive mechanism. Other senescence-inducing stimuli include radiation, genotoxic drugs, tissue injury and remodelling, and metabolic perturbations. Thus, both chemotherapy and radiotherapy induce a senescence response. Senescent cells remain chronically present, and they can promote local and systemic inflammation which has been associated with undesired secondary effects of the chemotherapies and tumor relapse.

Human PD-1 (hPD-1) was previously identified using a subtraction cloning based approach to select for proteins involved in apoptotic cell death (Yasumasa Ishida, Cells. 2020 June; 9 (6): 1376). hPD-1 is a member of the CD28/CTLA-4 family of molecules based on its ability to bind to PD-L1. Like CTLA4, hPD-1 is rapidly induced on the surface of T-cells in response to anti-CD3. In contrast to CTLA4, however, hPD-1 is also induced on the surface of B-cells (in response to anti-IgM). hPD-1 is also expressed on a subset of thymocytes and myeloid cells. hPD-1 polypeptides are inhibitory receptors capable of transmitting an inhibitory signal to an immune cell to thereby inhibit immune cell effector function, or are capable of promoting co-stimulation (e.g., by competitive inhibition) of immune cells, e.g., when present in soluble, monomeric form. An exemplary human PD-1 is shown in UniProtKB/Swiss-Prot Accession No. Q15116.

PD-1 has 2 ligands, programmed death ligand 1 (PD-L1) and programmed death ligand 2 (PD-L2). In cancer, PD-L1 and PD-L2 are expressed on the surface of tumor cells, promoting T-cell exhaustion. This suggests a role for PD-L1 and PD-L2 in tumor immune evasion. In the tumor microenvironment, T-cell exhaustion begins when repeated exposure to tumor antigen steadily increases hPD-1 activity: as uncontrolled hPD-1 signaling multiplies, T cells begin to lose their ability to respond. Over time, exhausted T cells become increasingly disabled and lose essential functions such as the ability to expand, fight tumor cells, and finally, survive. Tumor-infiltrating T cells across solid tumors and hematologic malignancies often show evidence of exhaustion, including: upregulation of hPD-1 and other inhibitors of immune function, decreased production of cytokines and cell-signaling molecules that help guide the immune response and impaired ability to kill tumor cells. An exemplary human PD-L2 is shown in UniProtKB/Swiss-Prot Accession No. Q9BQ51.

Chaib, “PD-L2 mediates immune evasion of chemotherapy-treated tumors and protects from tissue injury”, Doctoral Thesis, UNIVERSIDAD AUTÓNOMA DE MADRID, 2019 discloses that PD-L2 is upregulated by various cells upon senescence and damage induction. Moreover, it is disclosed that this upregulation leads to immune evasion post chemotherapy in the context of cancer, but that in the context of tissue injury this upregulation protects the host/organ for exaggerated tissue damage.

WO2021/197358 discloses a PD-L1 nano-antibody, a PD-L2 nano-antibody, and a bispecific antibody having both the PD-L1 nano-antibody and the PD-L2 nano-antibody. Said bispecific antibody has good binding activity to both PD-L1 and PD-L2 molecules and can block the interaction between PD-1 and PD-L1 and the interaction between PD-1 and PD-L2. It interacts and can simultaneously block the PD-L1/PD-1 and PD-L2/PD-1 signaling pathways in vitro and activate the expression of downstream reporter genes, thus having good anti-tumor activity. Furthermore, nanobodies only contain variable region of heavy chain and thus have no effector function. WO 2020/036635 discloses compositions for treating cancer in a subject comprising administering an effective amount of a nucleic acid encoding p53 and/or a nucleic acid encoding MDA-7 and at least one CD122 agonist and CD132 agonist to the subject. Such a CD122/CD132 agonist can be an IL-2/anti-IL-2 immune complex. None of these documents addresses the problem of senescent cells.

WO2019002581A1 discloses inhibitors of PD-1 or PD-L2 expression to eliminate damaged and/or senescent cells in a variety of diseases, including cancer. In addition, it describes that PD-L2 is highly expressed in senescent tumor cells and induces immunotolerance. However, the anti-tumor activity of said anti-PD-L2 agents is not satisfactory and there is no evidence that senescent cells can be eliminated.

The object of the present invention is to provide an agent for treating, ameliorating or preventing a disease or condition associated with the presence of senescent cells in cancer tissue, said agent promoting immune clearance of senescent cells in said tissue.

The object is solved by the antibody according to claim 1. Further preferred embodiments are subject of dependent claims 2 to 17.

In one aspect, the invention relates to an isolated monoclonal antibody that specifically binds to programmed cell death ligand 2 (PD-L2) and blocks activity against hPD-L2/hPD1 interaction, wherein said antibody comprises a heavy chain variable region and a light chain variable region, and comprises
a) as heavy chain variable region CDRs a CDRH1 region of SEQ ID NO: 13 (GYAFSNYFIE) or SEQ ID NO: 14 (GYSFSNYFIE); a CDRH2 region of SEQ ID NO: 15 (LNIPGSGGSNYAEKFKG); and a CDRH3 region of SEQ ID NO: 16 (RRLPPDWYFDV); and
b) as light chain region CDRs a CDRL1 region of SEQ ID NO: 17 (RSSQSLVHSGGNTYLH) or SEQ ID NO: 20 (RSSQSLVHSDGNTYLH); a CDRL2 region of SEQ ID NO: 18 (KVSNRFS); and a CDRL3 region of SEQ ID NO: 19 (SQSTHVPWT).

The antibodies of the present invention have a high affinity for human PD-L2 while also providing blocking activity against hPD1. In particular, the antibodies according to the present invention have a superior binding activity against human PD-L2 than commercially available monoclonal PD-L2 antibodies such as MIH-18, 24F.10C12, D7U86 and TY25. Moreover, it was shown, that they do not only effectively block the hPD-1/hPD-L2 interaction, but they can stimulate immune-mediated clearance of senescent cells in cancer tissue, and in particular senescent tumor cells. Thus, such senescent tumor cells are effectively recognised and eliminated by the immune system. Clearance of senescent cells can reduce the danger of metastasis promotion. In addition, it allows a full regression of the tumor concerned and reduces the risk of tumor relapse.

In a preferred embodiment of the present invention said antibody comprises a heavy chain variable region and a light chain variable region, and comprises as heavy chain variable region CDRs a CDRH1 region of SEQ ID NO: 14 (GYSFSNYFIE); CDRH2 region of SEQ ID NO: 15 (LNIPGSGGSNYAEKFKG); and CDRH3 region of SEQ ID NO: 16 (RRLPPDWYFDV); and as light chain region CDRs a CDRL1 region of SEQ ID NO: 17 (RSSQSLVHSGGNTYLH); a CDRL2 region of SEQ ID NO: 18 (KVSNRFS); and a CDRL3 region of SEQ ID NO: 19 (SQSTHVPWT), since said antibodies have a particularly high affinity for human PD-L2 while also providing blocking activity against hPD1. Furthermore, they have a low isomerization risk which is a significant advantage.

In another preferred embodiment the isolated monoclonal antibody comprises as heavy chain variable region CDRs a CDRH1 region of SEQ ID NO: 13 (GYAFSNYFIE); a CDRH2 region of SEQ ID NO: 15 (LNIPGSGGSNYAEKFKG); and a CDRH3 region of SEQ ID NO: 16 (RRLPPDWYFDV); and as light chain region CDRs a CDRL1 region of SEQ ID NO: 20 (RSSQSLVHSDGNTYLH); a CDRL2 region of SEQ ID NO: 18 (KVSNRFS); and a CDRL3 region of SEQ ID NO: 19 (SQSTHVPWT) since said antibodies have a particularly high affinity for human and/or mouse PD-L2, while also providing blocking activity against hPD1.

Within the context of the present invention an antibody typically comprises at least two heavy (H) chains and two light (L) chains interconnected by disulphide bonds. Each heavy chain is comprised of a heavy chain variable region (abbreviated herein as V_{H}) and a heavy chain constant region. The heavy chain constant region is comprised of three regions, C_{H}1, C_{H}2 and C_{H}3. Each light chain is comprised of a light chain variable region (abbreviated as VL) and a light chain constant region. The light chain constant region is comprised of one region, C_{L}. The V_{H} and V_{L} regions can be further subdivided into regions of hypervariability, termed Complementary Determining Regions (CDR), interspersed with regions that are more conserved, termed framework regions (FW). Each V_{H} and V_{L} is composed of three CDRs and four FWs, arranged from amino-terminus to carboxy-terminus in the following order: FW1, CDR1, FW2, CDR2, FW3, CDR3, FW4. The variable regions of the heavy and light chains contain a binding region that interacts with an antigen. The constant regions of the antibodies can mediate the binding of the immunoglobulin to host tissues and factors, including various cells of the immune system (e.g. effector cells) and the first component (C1q) of the classical complement system. The CDR regions of an Ig-derived region may be determined as described in Kabat “Sequences of Proteins of Immunological Interest”, 5th edit. NIH Publication no. 91-3242 U.S. Department of Health and Human Services (1991); Chothia J. Mol. Biol. 196 (1987), 901-917 or Chothia Nature 342 (1989), 877-883.

As used herein, the term “monoclonal antibody”, refers to an antibody which displays a single binding specificity and affinity for a particular epitope. Accordingly, the term “humanized monoclonal antibody” refers to an antibody which displays a single binding specificity, and which has variable and constant regions derived from human germline or non-germline immunoglobulin sequences.

The term “epitope,” as used herein, refers the portion or region of an antigenic molecule (e.g., a peptide), that is specifically bound by the antibody combining site of an antibody. An epitope typically includes at least 3, and more usually, at least 5 or 8 10 residues (e.g., amino acids or nucleotides). Typically, an epitope also is less than 20 residues (e.g., amino acids or nucleotides) in length, such as less than 15 residues or less than 12 residues. The term “epitope” encompasses both a linear epitope for which the consecutive amino acids are recognized by the antibody as well as a conformational epitope for which the antibodies recognize amino acids to the extent, they adopt a proper configuration or conformation. Consequently, in some epitopes, the conformation (three-dimensional structure) is as important as the amino acid sequence (primary structure).

As used herein, the term “isolated antibody” is intended to refer to an antibody which is substantially free of other antibodies having different antigenic specificities (e.g., an isolated antibody that specifically binds to PD-L2 and is substantially free of antibodies that do not bind to PD-L2). An isolated antibody that specifically binds to an epitope of human PD-L2 may, however, have cross-reactivity to other PD-L2 proteins from different species. However, in preferred embodiments, the antibody maintains higher affinity and selectivity for human PD-L2. In addition, an isolated antibody is typically substantially free of other cellular material and/or chemicals.

Within the context of the present invention the term “senescent cells in cancer tissue” includes various types of senescent cells in the tumor microenvironment, thus normal cells and tumor cells that have been induced into senescence. Senescent tumor cells originate from tumor cells, and are usually the common type senescent of cells in the tumor most microenvironment. They can be identified by knowns markers, for example by senescence-associated beta-galactosidase.

Within the context of the present invention VHP/VKP is the parental murine antibody on mIgG1 backbone, also called VH0/VK0-mIgG1, and VH0/VKP0 is the chimeric antibody on hIgG1 backbone. The variable regions are the same in VHP/VKP and VH0/VK0, thus VHP corresponds to VH0 and VKP corresponds to VK0. Preferably, the V region of the antibody of the present invention has sequences that are devoid of, or reduced in significant T cell epitopes. Eight heavy chains (VH0 (chimeric), VH1 to VH4 and VH6 to VH8 (all humanized)) and four light chains (VK0 (chimeric) and VK1 to VK3 (humanized)) sequences were found to provide particularly effective antibodies (Table 1).

VH0, VH1, VH2, VH3, VH4, VH6, VH7 and VH8 all comprise SEQ ID NO: 13 (GYAFSNYFIE) or SEQ ID NO: 14 (GYSFSNYFIE) as CDRH1, SEQ ID NO: 15 (LNIPGSGGSNYAEKFKG) as CDRH2; and SEQ ID NO: 16 (RRLPPDWYFDV) as CDRH3. VK0, VK1, VK2 and VK3 all comprise SEQ ID NO: 17 (RSSQSLVHSGGNTYLH) or SEQ ID NO: 20 (RSSQSLVHSDGNTYLH) as CDRL1; SEQ ID NO: 18 (KVSNRFS) as CDRL2; and SEQ ID NO: 19 (SQSTHVPWT) as CDRL3.

TABLE 1

| | |
|---|---|
| VH0; | QVQLQQSGAELVRPGTSVKVSCKASGYAFSNYFIEWVKQR |
| SEQ. ID | PGQGLEWIGLNIPGSGGSNYAEKFKGKATLTADKSSSTAY |
| NO. 1 | MQLSSLTSEDSAVYFCARRRLPPDWYFDVWGTGTTVTVSS |
| VH1 | QVQLVQSGAELKKPGSSVKVSCKASGYAFSNYFIEWVKQP |
| SEQ. ID | PGKGLEWIGLNIPGSGGSNYAEKFKGRATITADKSTSTAY |
| NO. 2 | MELSSLTSEDSAVYFCARRRLPPDWYFDVWGQGTTVTVSS |
| VH2 | QVQLVQSGAEVKKPGSSVKVSCKASGYAFSNYFIEWVKQP |
| SEQ. ID | PGKGLEWIGLNIPGSGGSNYAEKFKGRATITADKSTSTAY |
| NO. 3 | MELSSLRSEDSAVYFCARRRLPPDWYFDVWGQGTTVTVSS |
| VH3 | QVQLVQSGAEVKKPGSSVKVSCKASGYAFSNYFIEWVKQP |
| SEQ. ID | PGKGLEWIGLNIPGSGGSNYAEKFKGRVTITADKSTSTAY |
| NO. 4 | MELSSLRSEDSAVYFCARRRLPPDWYFDVWGQGTTVTVSS |
| VH4 | QVQLVQSGAEVKKPGSSVKVSCKASGYAFSNYFIEWVKQP |
| SEQ. ID | PGKGLEWIGLNIPGSGGSNYAEKFKGRVTITADKSTSTAY |
| NO. 5 | MELSSLRSEDTAVYYCARRRLPPDWYFDVWGQGTTVTVSS |
| VH6 | QVQLVQSGAEVKKPGSSVKVSCKASGYSFSNYFIEWVKQP |
| SEQ. ID | PGKGLEWIGLNIPGSGGSNYAEKFKGRVTITADKSTSTAY |
| NO. 6 | MELSSLRSEDTAVYYCARRRLPPDWYFDVWGQGTTVTVSS |
| VH7 | QVQLVQSGAELKKPGSSVKVSCKASGYAFSNYFIEWVKQP |
| SEQ. ID | PGKGLEWIGLNIPGSGGSNYAEKFKGRATITADKSTSTAY |
| NO. 7 | MELSSLRSEDTAVYYCARRRLPPDWYFDVWGQGTTVTVSS |
| VH8 | QVQLVQSGAEVKKPGSSVKVSCKASGYAFSNYFIEWVKQP |
| SEQ. ID | PGKGLEWIGLNIPGSGGSNYAEKFKGRATITADKSTSTAY |
| NO. 8 | MELSSLRSEDTAVYYCARRRLPPDWYFDVWGQGTTVTVSS |
| VK0 | DVVMTQTPLSLPVSLGDQASISCRSSQSLVHSDGNTYLHW |
| SEQ. ID | YLQKPGQSPKLLIYKVSNRFSGVPDRFSGSGSGTDFTLKI |
| NO. 9 | SRVEAEDLGVYFCSQSTHVPWTFGGGTKLEIK |
| VK1 | DVVMTQSPLSLPVTLGQPASISCRSSQSLVHSDGNTYLHW |
| SEQ. ID | YQQKPGQPPKLLIYKVSNRFSGVPDRESGSGSGTDFTLKI |
| NO. 10 | SRVEAEDVGVYFCSQSTHVPWTFGGGTKVEIK |
| VK2 | DVVMTQSPLSLPVTLGQPASISCRSSQSLVHSDGNTYLHW |
| SEQ. ID | YQQKPGQPPKLLIYKVSNRFSGVPDRFSGSGSGTDFTLKI |
| NO. 11 | SRVEAEDVGVYYCSQSTHVPWTFGGGTKVEIK |
| VK3 | DVVMTQSPLSLPVTLGQPASISCRSSQSLVHSGGNTYLHW |
| SEQ. ID | YQQKPGQPPKLLIYKVSNRFSGVPDRFSGSGSGTDFTLKI |
| NO. 12 | SRVEAEDVGVYFCSQSTHVPWTFGGGTKVEIK |
| VHP; | QVQLQQSGAELVRPGTSVKVSCKASGYAFSNYFIEWVKQR |
| SEQ. ID | PGQGLEWIGLNIPGSGGSNYAEKFKGKATLTADKSSSTAY |
| NO. 21 | MQLSSLTSEDSAVYFCARRRLPPDWYFDVWGTGTTVTVSS |
| VKP | DVVMTQTPLSLPVSLGDQASISCRSSQSLVHSDGNTYLHW |
| SEQ. ID | YLQKPGQSPKLLIYKVSNRFSGVPDRESGSGSGTDETLKI |
| NO. 22 | SRVEAEDLGVYFCSQSTHVPWTFGGGTKLEIK |

Preferably, the isolated monoclonal antibody comprises: (a) a heavy chain variable region having a complementarity determining region 1 (CDRH1) comprising the amino acid sequence of SEQ ID NO: 13 (GYAFSNYFIE) or SEQ ID NO: 14 (GYSFSNYFIE); a CDRH2 comprising the amino acid sequence of SEQ ID NO: 15 (LNIPGSGGSNYAEKFKG); and a CDRH3 comprising the amino acid sequence of SEQ ID NO: 16 (RRLPPDWYFDV), wherein the remaining part of the heavy chain sequence has at least 95% identity to a heavy chain sequence selected from the group consisting of the sequences listed in Table 1; and
(b) a light chain variable region having a CDRL1 comprising the amino acid sequence of SEQ ID NO: 17 (RSSQSLVHSGGNTYLH) or SEQ ID NO: 20 (RSSQSLVHSDGNTYLH); a CDRL2 comprising the amino (KVSNRFS); and a CDRL3 acid sequence of SEQ ID NO: 18 comprising the amino acid sequence of SEQ ID NO: 19 (SQSTHVPWT), wherein the remaining part of the heavy chain sequence has at least 95% identity to a heavy chain sequence selected from the group consisting of the sequences listed in Table 1.

As shown in Table 2 all humanized variant antibodies containing VH1, VH2, VH3, VH4, VH6, VH7 and VH8 as heavy chains and VK1, VK2 and VK3 as light chains bind human PD-L2 antigen with high affinity.

Table 2: Single cycle kinetic parameters of chimeric (VH0/VK0) and humanized variants (tested as cell culture supernatants) binding to human PD-L2 antigen as determined using the Biacore 8K. As used herein, the term “K_{D}” is intended to refer to the dissociation equilibrium constant of a particular antibody-antigen interaction, ka is the binding rate constant and kd is the dissociation rate constant. The binding affinity of antibodies of the disclosed invention may be measured or determined by standard antibody-antigen assays. The relative K_{D} was calculated by dividing the K_{D} of the humanized variant by that of the VH0/VK0 assayed in the same experiment.

TABLE 2

| Variant | ka (1/Ms) | kd (1/s) | K_{D} (M) | Relative K_{D} |
|---|---|---|---|---|
| VH0 VK0 | 2.72 × 10⁻⁶ | 9.72 × 10⁻⁵ | 3.58 × 10⁻¹¹ | 1.00 |
| SEQ ID NO: 1/SEQ ID NO: 9 | | | | |
| VH0 VK1 | 2.60 × 10⁻⁶ | 9.88 × 10⁻⁵ | 3.79 × 10⁻¹¹ | 1.06 |
| SEQ ID NO: 1/SEQ ID NO: 10 | | | | |
| VH1 VK0 | 2.56 × 10⁻⁶ | 1.09 × 10⁻⁴ | 4.27 × 10⁻¹¹ | 1.19 |
| SEQ ID NO: 2/SEQ ID NO: 9 | | | | |
| VH1 VK1 | 2.32 × 10⁻⁶ | 1.10 × 10⁻⁴ | 4.75 × 10⁻¹¹ | 1.33 |
| SEQ ID NO: 2/SEQ ID NO: 10 | | | | |
| VH1 VK2 | 2.39 × 10⁻⁶ | 1.16 × 10⁻⁴ | 4.87 × 10⁻¹¹ | 1.36 |
| SEQ ID NO: 2/SEQ ID NO: 11 | | | | |
| VH1 VK3 | 1.64 × 10⁻⁶ | 3.65 × 10⁻⁴ | 2.23 × 10⁻¹⁰ | 6.23 |
| SEQ ID NO: 2/SEQ ID NO: 12 | | | | |
| VH2 VK1 | 2.17 × 10⁻⁶ | 1.19 × 10⁻⁴ | 5.51 × 10⁻¹¹ | 1.54 |
| SEQ ID NO: 3/SEQ ID NO: 10 | | | | |
| VH2 VK2 | 2.19 × 10⁻⁶ | 1.22 × 10⁻⁴ | 5.55 × 10⁻¹¹ | 1.55 |
| SEQ ID NO: 3/SEQ ID NO: 11 | | | | |
| VH2 VK3 | 1.55 × 10⁻⁶ | 4.14 × 10⁻⁴ | 2.67 × 10⁻¹⁰ | 7.46 |
| SEQ ID NO: 3/SEQ ID NO: 12 | | | | |
| VH3 VK1 | 2.11 × 10⁻⁶ | 1.28 × 10⁻⁴ | 6.06 × 10⁻¹¹ | 1.69 |
| SEQ ID NO: 4/SEQ ID NO: 10 | | | | |
| VH3 VK2 | 2.19 × 10⁻⁶ | 1.32 × 10⁻⁴ | 6.04 × 10⁻¹¹ | 1.69 |
| SEQ ID NO: 4/SEQ ID NO: 11 | | | | |
| VH3 VK3 | 1.59 × 10⁻⁶ | 4.97 × 10⁻⁴ | 3.14 × 10⁻¹⁰ | 8.77 |
| SEQ ID NO: 4/SEQ ID NO: 12 | | | | |
| VH4 VK1 | 2.13 × 10⁻⁶ | 1.25 × 10⁻⁴ | 5.86 × 10⁻¹¹ | 1.64 |
| SEQ ID NO: 5/SEQ ID NO: 10 | | | | |
| VH4 VK2 | 2.17 × 10⁻⁶ | 1.29 × 10⁻⁴ | 5.96 × 10⁻¹¹ | 1.66 |
| SEQ ID NO: 5/SEQ ID NO: 11 | | | | |
| VH4 VK3 | 1.62 × 10⁻⁶ | 4.66 × 10⁻⁴ | 2.87 × 10⁻¹⁰ | 8.02 |
| SEQ ID NO: 5/SEQ ID NO: 12 | | | | |
| VH6 VK1 | 2.29 × 10⁻⁶ | 1.21 × 10⁻⁴ | 5.29 × 10⁻¹¹ | 1.48 |
| SEQ ID NO: 6/SEQ ID NO: 10 | | | | |
| VH6 VK2 | 2.18 × 10⁻⁶ | 1.28 × 10⁻⁴ | 5.88 × 10⁻¹¹ | 1.64 |
| SEQ ID NO: 6/SEQ ID NO: 11 | | | | |
| VH6 VK3 | 1.69 × 10⁻⁶ | 4.44 × 10⁻⁴ | 2.62 × 10⁻¹⁰ | 7.32 |
| SEQ ID NO: 6/SEQ ID NO: 12 | | | | |
| VH7 VK1 | 2.27 × 10⁻⁶ | 1.13 × 10⁻⁴ | 4.99 × 10⁻¹¹ | 1.39 |
| SEQ ID NO: 7/SEQ ID NO: 10 | | | | |
| VH7 VK2 | 2.26 × 10⁻⁶ | 1.18 × 10⁻⁴ | 5.23 × 10⁻¹¹ | 1.46 |
| SEQ ID NO: 7/SEQ ID NO: 11 | | | | |
| VH7 VK3 | 1.55 × 10⁻⁶ | 3.44 × 10⁻⁴ | 2.22 × 10⁻¹⁰ | 6.20 |
| SEQ ID NO: 7/SEQ ID NO: 12 | | | | |
| VH8 VK1 | 2.26 × 10⁻⁶ | 1.20 × 10⁻⁴ | 5.30 × 10⁻¹¹ | 1.48 |
| SEQ ID NO: 8/SEQ ID NO: 10 | | | | |
| VH8 VK2 | 2.29 × 10⁻⁶ | 1.21 × 10⁻⁴ | 5.28 × 10⁻¹¹ | 1.47 |
| SEQ ID NO: 8/SEQ ID NO: 11 | | | | |
| VH8 VK3 | 1.59 × 10⁻⁶ | 3.88 × 10⁻⁴ | 2.44 × 10⁻¹⁰ | 6.82 |
| SEQ ID NO: 8/SEQ ID NO: 12 | | | | |

As mentioned above VH0/VK0 corresponds to the chimeric antibody (mouse Fab on human IgG1 backbone) and VHP/VKP is parental mouse antibody (mouse Fab on murine IgG1 backbone).

Preferably, the monoclonal antibody of the present invention comprises a heavy chain variable region and a light chain variable region, and:
(a) the heavy chain variable region comprises the amino acid sequence of SEQ ID NO:1 (VH0) and the light chain variable region comprises the amino acid sequence of SEQ ID NO: 9 (VK0); or
(b) the heavy chain variable region comprises the amino acid sequence of SEQ ID NO: 6 (VH6) and the light chain variable region comprises the amino acid sequence of SEQ ID NO: 10 (VK1), of SEQ ID NO: 11 (VK2) or of SEQ ID NO: 12 (VH3); or
(c) the heavy chain variable region comprises the amino acid sequence of SEQ ID NO: 7 (VH7) and the light chain variable region comprises the amino acid sequence of SEQ ID NO: 11 (VK2); or
(d) the heavy chain variable region comprises the amino acid sequence of SEQ ID NO: 8 (VH8) and the light chain variable region comprises the amino acid sequence of SEQ ID NO: 11 (VK2).

Based upon expression, iTope™-AI scores and TCEM™ (in silico tools provided by Abzena that rapidly screen antibodies and proteins for potential immunogenicity), the above mentioned chimeric VH0/VK0 antibody (SEQ ID NO: 1/SEQ ID NO: 9) and six humanized antibodies VH4/VK2 (SEQ ID NO: 5/SEQ ID NO: 11), VH6/VK3 (SEQ ID NO: 6/SEQ ID NO: 12), VH6/VK1 (SEQ ID NO: 6/SEQ ID NO: 10), VH6/VK2 (SEQ ID NO: 6/SEQ ID NO: 11), VH7/VK2 (SEQ ID NO: 7/SEQ ID NO:11) and VH8/VK2 (SEQ ID NO: 7/SEQ ID NO: 11) are especially preferred. The variant VH6/VK3 (SEQ ID NO: 6/SEQ ID NO: 12) has the advantage that there is no isomerisation risk which might occur with the other 5 preferred humanized variants VH4/VK2 (SEQ ID NO: 5/SEQ ID NO: 11), VH6/VK1 (SEQ ID NO: 6/SEQ ID NO:10), VH6/VK2 (SEQ ID NO: 6/SEQ ID NO: 11), VH7/VK2 (SEQ ID NO: 7/SEQ ID NO: 11) and VH8/VK2 (SEQ ID NO: 7/SEQ ID NO:11).

Table 3 shows Ka, Kd, K_{D} and relative K_{D} values for preferred embodiments by Biacore:

TABLE 3

| Variant | ka (1/Ms) | kd (1/s) | K_{D} (M) | Relative K_{D} |
|---|---|---|---|---|
| VH0/VK0 | 2.72 × 10⁻⁶ | 9.72 × 10⁻⁵ | 3.58 × 10⁻¹¹ | 1.00 |
| SEQ ID NO: 1/SEQ ID NO: 9 | | | | |
| VH4/VK2 | 2.17 × 10⁻⁶ | 1.29 × 10⁻⁴ | 5.96 × 10⁻¹¹ | 1.66 |
| SEQ ID NO: 5/SEQ ID NO: 11 | | | | |
| VH6/VK1 | 2.29 × 10⁻⁶ | 1.21 × 10⁻⁴ | 5.29 × 10⁻¹¹ | 1.48 |
| SEQ ID NO: 6/SEQ ID NO: 10 | | | | |
| VH6/VK2 | 2.18 × 10⁻⁶ | 1.28 × 10⁻⁴ | 5.88 × 10⁻¹¹ | 1.64 |
| SEQ ID NO: 6/SEQ ID NO: 11 | | | | |
| VH6/VK3 | 1.69 × 10⁻⁶ | 4.44 × 10⁻⁴ | 2.62 × 10⁻¹⁰ | 7.32 |
| SEQ ID NO: 6/SEQ ID NO: 12 | | | | |
| VH7/VK2 | 2.26 × 10⁻⁶ | 1.18 × 10⁻⁴ | 5.23 × 10⁻¹¹ | 1.46 |
| SEQ ID NO: 7/SEQ ID NO: 11 | | | | |
| VH8/VK2 | 2.29 × 10⁻⁶ | 1.21 × 10⁻⁴ | 5.28 × 10⁻¹¹ | 1.47 |
| SEQ ID NO: 8/SEQ ID NO: 11 | | | | |

In one aspect, the isolated antibody according to the present invention binds to human recombinant PD-L2 with a dissociation constant (K_{D}) equal to or less than 10⁻¹⁰ M.

In another aspect, the isolated antibody according to the present invention blocks in vitro the interaction between human programmed cell death protein 1 (PD-1) and human PD-L2 with an EC₅₀ (half maximal effective concentration) equal to or less than 1.3 nM in a bioluminescent cell-based assay using Jurkat T cells expressing human PD-1 and a luciferase reporter driven by an NFAT response element (NFAT-RE) and CHO-K1 cells expressing human PD-L2 and an engineered cell surface protein designed to activate cognate TCRs in an antigen-independent manner. Such antibodies have a high clinical efficacy.

FIG. 1A shows that the antibodies according to the present invention (EC₅₀=0.219 nM) bind cellular PDL2 better than commercial antibodies MIH-18 (EC₅₀=0.618 nM), 24F.10C12 (EC₅₀=0.70 nM), and TY25. Blocking activity is specifically shown in FIGS. 1B and 1E, where recombinant hPD-1 ligand is added to CHOK-1 cells overexpressing hPD-L2 by flow cytometry. FIG. 1C shows the blocking activity of VH0/VK0 and 5 humanized variants and MIH-18 against cellular hPD-L2/hPD-1 interaction as measured by a fluorescent reporter and FIG. 1D shows that VH0/VK0-mIgG1 and VH6/VK6 have superior blocking activities against cellular hPD-L2/hPD-1 interaction compared to commercial antibodies MIH-18, 24F.10C12, and D7U86.

The disclosed experimental data of FIGS. 1A to 1E clearly indicate that the chimeric antibody VH0/VK0 (SEQ ID NO: 1/SEQ ID NO: 9) and the five humanized antibodies VH6/VK3 (SEQ ID NO: 6/SEQ ID NO: 12), VH6/VK1 (SEQ ID NO: 6/SEQ ID NO: 10), VH6/VK2 (SEQ ID NO: 6/SEQ ID NO: 11), VH7/VK2 (SEQ ID NO: 7/SEQ ID NO: 11) and VH8/VK2 (SEQ ID NO: 7/SEQ ID NO:11) have similar physiological properties regarding the interaction with hPD-L2. In particular, it is shown that the difference of one amino acid at position 3 of CDRH1 (A vs S) and the difference of one amino acid at position 10 (G vs D) of CDRL1 does not negatively impact the physiological properties. Especially good results could be obtained with VH0/VK0 (SEQ ID NO:1/SEQ ID NO: 9), VH6/VK3 (SEQ ID NO: 6/SEQ ID NO: 12), VH6/VK2 (SEQ ID NO: 6/SEQ ID NO: 11) and VH7/VK2 (SEQ ID NO: 7/SEQ ID NO:11). In another aspect, the isolated antibody according to the present invention specifically binds both to human and murine PD-L2.

FIGS. 2A and 2B show that parental and humanized variant antibodies according to the present invention have a superior binding activity for recombinant and cellular mPD-L2 than the commercially available anti-mPD-L2 TY-25. FIG. 2C shows cross-reactivity of the antibody according to the present invention to recombinant mPD-L2 by ELISA, whereas commercially available MIH-18 shows no cross-reactivity to recombinant mPD-L2. Especially good results could be obtained with VH0/VK0-mIgG1.

In another aspect, the isolated antibody according to the present invention binds to human PD-L2 with a dissociation constant (K_{D}) equal to or less than 10⁻¹⁰ M and at the same time blocks in vitro the interaction between human programmed cell death protein 1 (PD-1) and human PD-L2 with an EC₅₀ equal to or less than 1.3 nM, equal to or less than 0.65 nM, in a bioluminescent cell-based assay using Jurkat T cells expressing human PD-1 and a luciferase reporter driven by an NFAT response element (NFAT-RE) and CHO-K1 cells expressing human PD-L2 and an engineered cell surface protein designed to activate cognate TCRs in an antigen-independent manner.

In another aspect, the isolated antibody according to the present invention binds to human PD-L2 with a dissociation constant (K_{D}) equal to or less than 10⁻¹⁰ M and specifically binds both to human and murine PD-L2 which eases pre-clinical testing.

In another aspect, the isolated antibody according to the present invention binds to human PD-L2 with a dissociation constant (K_{D}) equal to or less than 10⁻¹⁰ M and at the same time blocks in vitro the interaction between human Programmed cell death protein 1 (PD-1) and human PD-L2 with an EC₅₀ equal to or less than 1.3 nM in a bioluminescent cell-based assay using Jurkat T cells expressing human PD-1 and a luciferase reporter driven by an NFAT response element (NFAT-RE) and CHO-K1 cells expressing human PD-L2 and an engineered cell surface protein designed to activate cognate TCRs in an antigen-independent manner and specifically binds both to human and murine PD-L2.

The antibodies according to the present invention have a superior binding activity against cellular hPD-L2 and blocking activity against the cellular hPD-1/hPD-L2 interaction compared to a panel of commercially available anti-PD-L2 antibodies (see FIG. 1a). Furthermore, the antibodies according to the present invention showed a higher blocking activity than MIH-18, 24F.10C12, D7U86 and associated isotype negative controls against cellular hPD-L2/hPD-1 interaction as measured by fluorescent reporter in hPD-1 expressing Jurkat cells co-cultured with hPD-L2 expressing CHOK-1 cells (FIG. 1b). In still another embodiment, the isolated monoclonal antibody is chimeric, humanized or human.

The term “chimeric antibody” means an antibody having light and heavy chain genes which have been constructed, typically by genetic engineering, from immunoglobulin variable and/or constant region genes belonging to one or more different species. For example, the variable segments of the genes (or, e.g., one or more of the complementarity determining regions (CDRs) within the variable regions) from, e.g., a mouse antibody (e.g., a monoclonal or polyclonal antibody), may be used in conjunction with human constant segments to produce the chimeric antibody. A therapeutic chimeric antibody according to the present invention is thus a hybrid protein composed of the variable or antigen-binding region from a mouse antibody (e.g., one or more of the CDRs of a mouse antibody) and the constant or effector region from a human antibody, although other mammalian species may be used. The chimeric antibody can also include amino acid sequence obtained from a protein source other than an antibody.

The term “humanized antibody” means a type of chimeric antibody comprising a human framework region and one or more CDRs from a non-human (usually a mouse or rat) immunoglobulin. The non-human immunoglobulin providing the CDRs is called the “donor” and the human immunoglobulin providing the framework is called the “acceptor”. Constant regions need not be present, but if they are, they should be substantially identical to human immunoglobulin constant regions, i.e., at least about 85-90%, preferably about 95% or more identical. Hence, all parts of a humanized immunoglobulin, except possibly the CDRs, are substantially identical to corresponding parts of natural human immunoglobulin sequences. A humanized antibody is useful as an effective component in a therapeutic agent according to the present invention since antigenicity of the humanized antibody in the human body is lowered.

The term “human antibody” means that the amino acid sequence of the antibody is fully human, i.e., human heavy and light chain variable and constant regions.

In another aspect, a pharmaceutical composition comprising an isolated monoclonal antibody according to the present invention and a pharmaceutically acceptable excipient, is provided.

In another aspect, an isolated nucleic acid sequence that encodes the amino acid sequence of the light chain variable region and/or the heavy chain variable region of the antibody according to the present invention, is provided. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art.

In another aspect, a recombinant expression vector comprising such a nucleic acid sequence is provided. As used herein, the term “vector” is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

In another aspect, an isolated host cell which comprises the recombinant expression vector described herein and/or expresses the antibody thereof described herein, is provided. As used herein, the term “isolated host cell” (or simply “host cell”), is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

Preferably, the antibody according to the present invention is used in treating, ameliorating or preventing a disease or condition associated to the presence of senescent cells in cancer tissue, preferably selected from the group consisting of senescent stromal cells, immune infiltrating cells and senescent tumor cells, most preferably senescent tumor cells.

In a preferred embodiment, the antibody according to the present invention is used in treating, ameliorating or preventing a disease or condition associated to the presence of senescent cells in cancer tissue, and in particular of senescent tumor cells, wherein the antibody is administered in combination with a chemotherapeutic agent. The combination of the antibody according to the present invention and the chemotherapeutic agent results in a synergistic inhibition of tumor growth. In addition, the immune clearence of the senescent cells is stimulated. A “chemotherapeutic agent” is a chemical compound useful in the treatment of cancer. Examples of chemotherapeutic agents include is a chemical compound useful in the treatment of cancer. Preferably, the chemotherapeutic agent is selected from the group consisting of alkylating agents, nitrosourea agents, antimetabolites, antitumor antibiotics, alkaloids derived from plant, topoisomerase inhibitors, hormone therapy medicines, hormone antagonists, aromatase inhibitors, P-glycoprotein inhibitors, platinum complex derivatives, growth inhibitory agents, radioactive isotopes, target agents, in particular CDK4/6 inhibitors, other immunotherapeutic drugs, and other anticancer agents and combinations thereof.

More specifically, the chemotherapeutic agent is selected from the group consisting of danorubicin, etoposide, mitoxantrone, camptothecin, irinotecan, topotecan, fusulfan, temozolomide, carmustine, dacarbazine, cyclophosphamide, melphalan, mitomycin C, cisplatin, carboplatin, oxaliplatin, methotrexate, pemetrexed, gemcitabine, azacitdine, bromodeoxyuridine, 5-fluorouracil, mycophenolic acid, hydroxyurea, antinomycin D, paclitaxel, docaetaxel, vincristine, vinblastine, tamoxifen, fulvestrant, androgen deprivation), most preferably doxorubicine.

FIGS. 3A and 3B show that PyMT mice treated with the antibody according to the present invention in combination with a chemotherapeutic agent and in particular with doxorubicin had smaller progression of tumors and smaller tumor volumes compared to tumor in mice treated with TY-25 in combination with doxorubicin. Furthermore, as shown in FIGS. 3C and 3D doxorubicin significantly induces double-positive cells expressing both SA-beta galactosidase and PD-L2 and that a co-treatment with the antibody according to the present invention reduces this senescent cell burden. The final tumor volumes after four weeks of PyMT mice treated with either 4 mg/kg doxorubicin alone once a week, or the combination of 4 mg/kg of doxorubicin once a week in combination with 3 weekly administrations is significantly reduced when administering the antibody according to the present invention when compared to the commercially available TY-25 (FIG. 3F). In addition, it was shown that the number of remaining senescent cells according to SA-β-Gal staining in PyMT tumors significantly decreased after treating mice with the antibody according to the present invention (FIG. 3G). Thus, the antibody according to the present invention favors immune clearance of chemotherapy-treated cancer cells. It was demonstrated that PyMT spontaneous tumors show increased relative numbers of CD3+, CD8+ and CD4+ cells when PyMT mice are treated with the combination of doxorubicin and the antibody according to the present invention, compared to vehicle or doxorubicin-only treated mice. Said finding is consistent with PD-L2's function as a negative immune checkpoint inhibitor for T cells (FIG. 3D), i.e. it specifically reduces or turns down immune signals. Furthermore, the combination treatment of doxorubicin and the antibody according to the present invention inhibited tumor growth more than treatment of doxorubicin and mIgG1 control in the PyMT spontaneous model (FIG. 3E). Administration of depleting antibodies against CD8+ cells were able to relieve the inhibition of doxorubicin and the antibody of the present invention on tumor progression, while depleting antibodies against CD4+ cells did not relieve this inhibition, suggesting a dominant role for CD8+ in the mechanism of action of the antibody according to the present invention.

Preferably, the antibody according to the present invention comprises the FC region effector function since they provide a higher anti-tumor activity. As shown in FIG. 4, tumor volumes of PyMT mice treated with either 4 mg/kg doxorubicin alone once a week, or the combination of 4 mg/kg of doxorubicin once a week in combination with 3 weekly administrations of either VH0/VK0-mIgG2a show a higher anti-tumor activity, whereas antibodies having effector reduced VH0/VK0-mIgG2a/LALA or effector deficient VH0/VK0-mIgG2a/LALAPG over four weeks showed a lower anti-tumor activity. Target agents are preferably particular CDK4/6 inhibitors, and most preferably selected from the group consisting of imatinib, nilotinib, trametinib, vemurafenib, dasatinib, lapatinib, neratinib, afatinib, getfinib, erlotinib, sorafenib, sirolimus, rituximab, obinutuzumab, pertuzumab, trastuzumab, bevacizuumab, ranibizumab, palbociclib, ademaciclib, ribociclib, olaparib, niraparib, rucaparib and bortezomib.

Most preferably, the antibody according to the present invention is used in treating, ameliorating or preventing a disease or condition associated to the presence of senescent cells, wherein the disease or condition is cancer. As used herein, the terms “cancer” or “tumor” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. As used herein, the term “cancer” includes premalignant as well as malignant cancers.

Most preferably, the cancer is selected from the group consisting of breast cancer, non-small cell lung cancer, ovarian cancer, head squamous cell carcinoma, neck squamous cell carcinoma, carcinoma, squamous carcinoma in cervical canal, eyelid, tunica conjunctiva, vagina, lung, oral cavity, skin, urinary bladder, tongue, larynx or gullet, adenocarcinoma in prostate, small intestine, endometrium, cervical canal, large intestine, pancreas, gullet, intestinum rectum, uterus, stomach, mammary glandovary, sarcomata in myogenic sarcoma, leukosis, neuroma, melanoma, and lymphoma, most preferably selected from the group consisting of breast cancer, non-small cell lung cancer, ovarian cancer, head squamous cell carcinoma and neck squamous cell carcinoma.

### FIGURES

FIG. 1: shows VH0/VK0-mIgG1 has superior binding activity against recombinant and cellular hPD-L2 and blocking activity against the cellular hPD-1/hPD-L2 interaction compared to a panel of commercially available anti-PD-L2 antibodies, wherein

FIG. 1A) shows relative binding of VH0/VK0-mIgG1 versus MIH-18 and 24F.10C12 (all commercially available antibodies known to hPD-L2 in different applications), TY-25 (a commercially antibody known to bind mPD-L2 in different applications), and associated isotype negative controls by ELISA with recombinant hPD-L2.

FIG. 1B) shows binding activity of VH0/VK0-mIgG1 versus MIH-18, 24F.10C12, D7U86, TY-25 and associated isotype negative controls against cellular hPD-L2 expressed on CHOK-1 cells;

FIG. 1C: shows the blocking activity of VH0/VK0, VH6/VK1, VH6/VK2, VH6/VK3, VH7/VK2, and VH8/VK2 and MIH-18 against cellular hPD-L2/hPD-1 interaction as measured by fluorescent reporter in hPD-1 expressing Jurkat cells co-cultured with hPD-L2 expressing CHO-K1 cells;

FIG. 1D: shows that VH0/VK0-mIgG1 and VH6/VK6 have superior blocking activities against cellular hPD-L2/hPD-1 interaction compared to MIH-18, 24F.10C12, and D7U86 as measured by fluorescent reporter in hPD-1 expressing Jurkat cells co-cultured with hPD-L2 expressing CHO-K1 cells;

FIG. 1E: shows that VH0/VK0-mIgG1 has superior blocking activity against recombinant hPD-1 binding to CHOK-1 cell expressing hPD-L2 compared to a panel of commercially available anti-PD-L2 antibodies;

FIG. 2: Parental and humanized variant anti-PD-L2 antibodies, compared to commercially available anti-mPD-L2 TY-25, have superior binding activity for recombinant and cellular mPD-L2, wherein

FIG. 2A): shows PyMT mice treated with doxorubicin in combination with 3 mg/kg VH0/VK0-mIgG1 over four weeks yields a greater reduction in final tumor volume compared to doxorubicin alone or the combination of TY-25 and doxorubicin

FIG. 2B): shows the relative binding of VH0/VK0 and VH8/VK2 versus TY-25 to CHO-K1 cells overexpressing mPD-L2 by flow cytometry;

FIG. 2C): VH0/VK0-mIgG1 shows cross-reactivity to recombinant mPD-L2 by ELISA, whereas commercially available MIH-18 shows no cross-reactivity to recombinant mPD-L2, similar to isotype control mIgG1

FIG. 3: shows that VH0/VK0-mIgG1 has superior anti-tumor activity in combination with doxorubicin compared to TY-25 and demonstrates senescence-clearing activity to immune system activity in the murine PyMT breast cancer model, wherein

FIG. 3A) shows mice in the implantable PyMT model, treated with VH0/VK0-mIgG1 in combination with doxorubicin had smaller progression of tumors up to day 49 compared to tumor in mice treated with TY-25 in combination with doxorubicin. Error bars represent SEM

FIG. 3B) shows immunohistochemistry double-staining for senescence marker SA-β-galactosidase in combination with PD-L2 shows that doxorubicin significantly induces double-positive cells and that treatment with VH0/VK0-mIgG1 effectively reduces this senescent cell burden.

FIG. 3C) shows quantification of cells double-positive for PD-L2 and SA-βGal in PyMT tumors shows reduction of senescent cell burden when animals are co-treated with VH0/VK0-mIgG1. Error bars represent SEM and values for group treated with Doxorubicin+VH0/VK0-mIgG1 is significantly lower than groups treated with Vehicle or Doxorubicin+mIgG1 (p<0.05 by Student's t-test).

FIG. 3D) shows PyMT spontaneous tumors show increased relative numbers of CD3+, CD8+ and CD4+ cells when PyMT mice are treated with the combination of doxorubicin and VH0/VK0-mIgG1, compared to vehicle or doxorubicin-only treated mice, consistent with PD-L2's function as a negative immune checkpoint inhibitor for T cells. Error bars represent SEM and values for group treated with Doxorubicin+VH0/VK0-mIgG1 is significantly greater than groups treated with Vehicle or Doxorubicin+mIgG1 for CD3, CD8 and CD4 (p<0.05 by Student's t-test)

FIG. 3E) shows the combination treatment of doxorubicin and VH0/VK0-mIgG1 inhibited tumor growth more than treatment of doxorubicin and mIgG1 control in the PyMT spontaneous model. Administration of depleting antibodies against CD8+ cells were able to relieve the inhibition of doxorubicin and VH0/VK0-mIgG1 on tumor progression, while depleting antibodies against CD4+ cells only slightly relieved this inhibition, suggesting a dominant role for CD8+ in the mechanism of action of VH0/VK0-mIgG1. Error bars represent SEM and values for group treated with Doxorubicin+VH0/VK0-mIgG1 is significantly greater than group treated with Doxorubicin+mIgG1 or Doxorubicin+VH0/VK0-mIgG1+anti-CD8 (p<0.05 by Student's t-test9;

FIG. 3F) shows quantification of remaining senescent cells according to SA-βGal staining in PyMT tumors after treating mice as in A

FIG. 3G) Representative images of SA-β-Gal staining in PyMT tumors after treating mice as in 2A

FIG. 4: shows that the FC region effector function is required for full anti-tumor activity of anti-PD-L2 antibodies containing the VH0/VK0 variable regions in spontaneous PyMT model. Final tumor volumes of PyMT mice treated with either 4 mg/kg doxorubicin alone once a week, or the combination of 4 mg/kg of doxorubicin once a week in combination with 3 weekly administrations of 10 mg/Kg of either VH0/VK0-mIgG2a or effector reduced VH0/VK0-mIgG2a/LALA or effector deficient VH0/VK0-mIgG2a/LALAPG over four weeks.

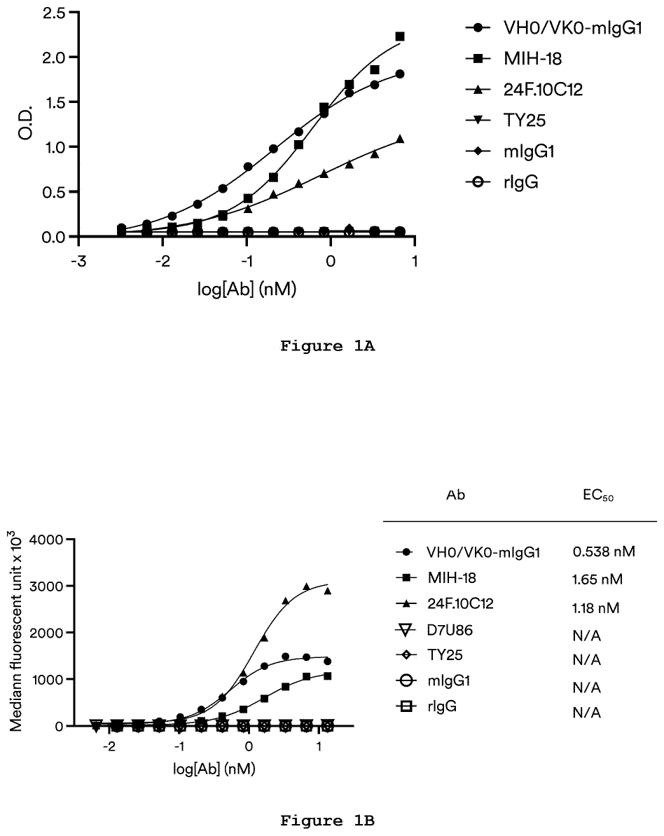

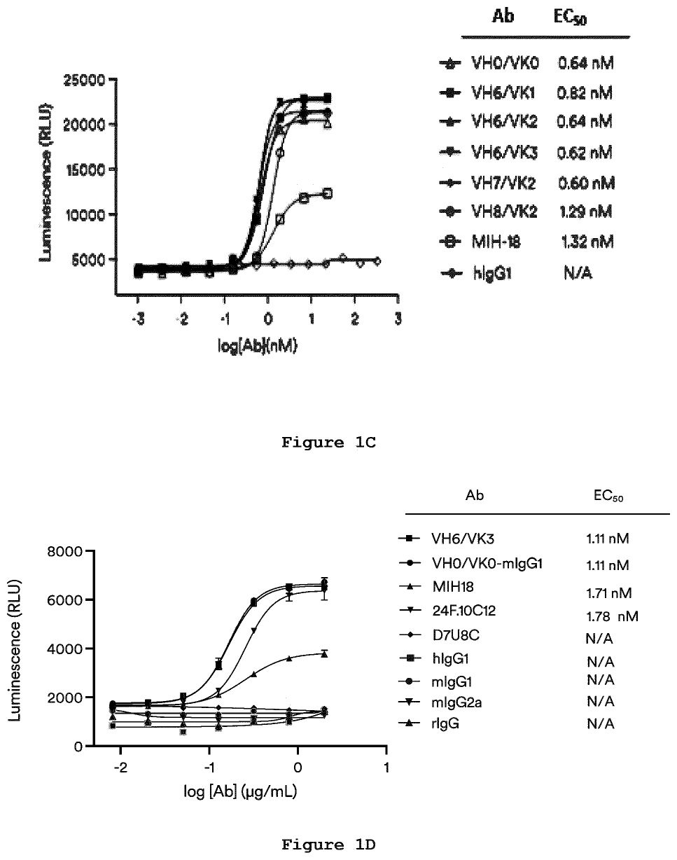

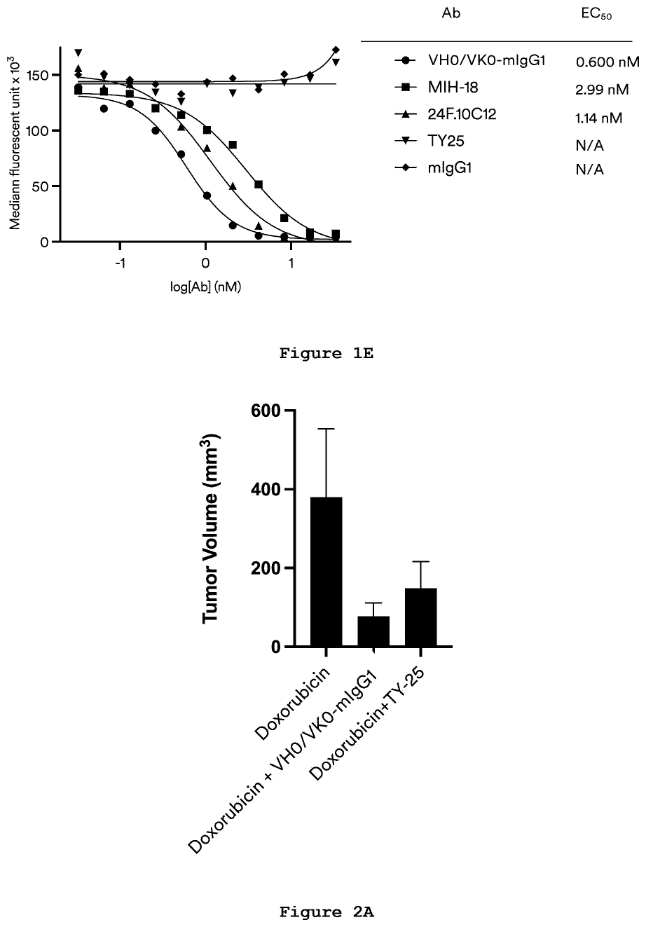

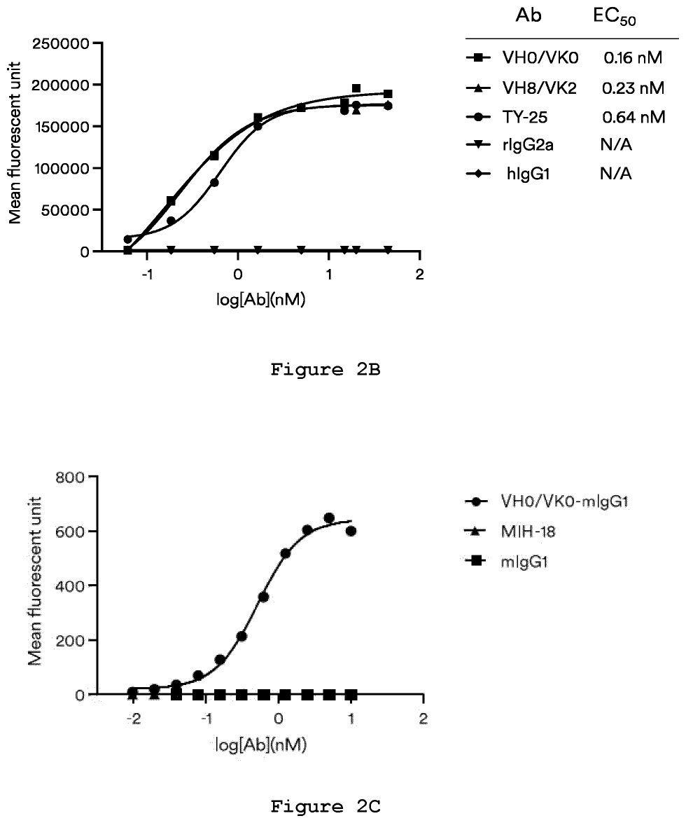

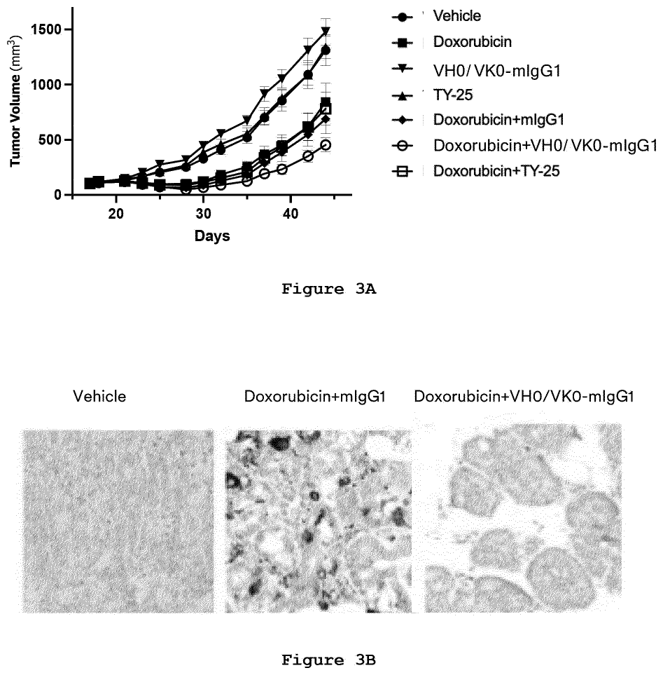

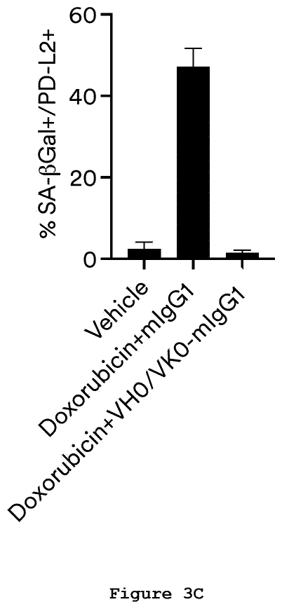

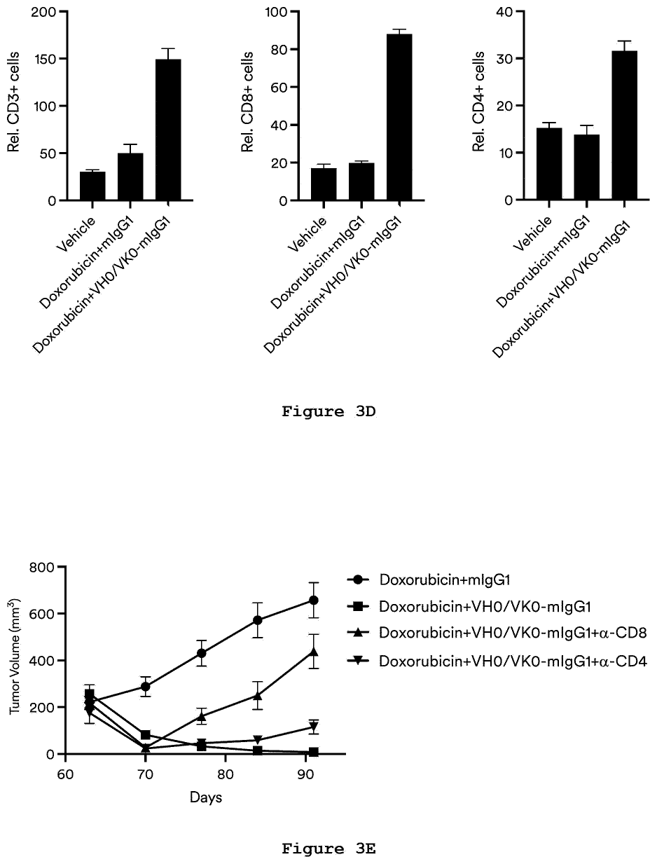

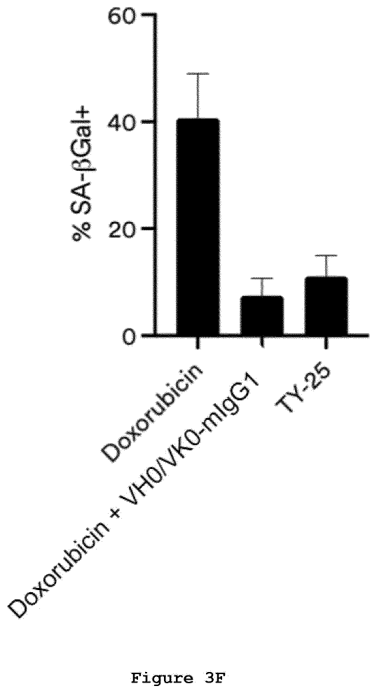

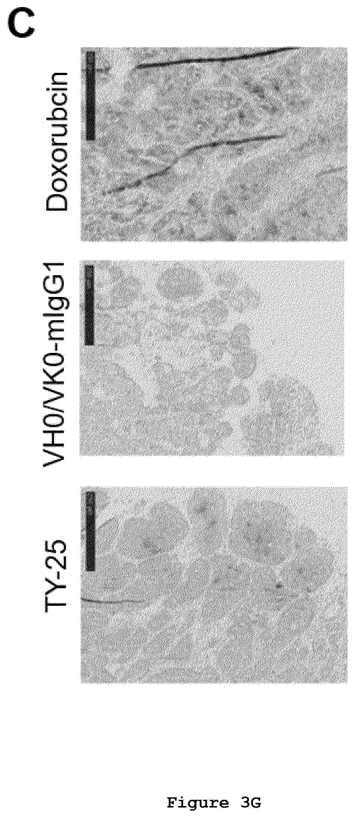

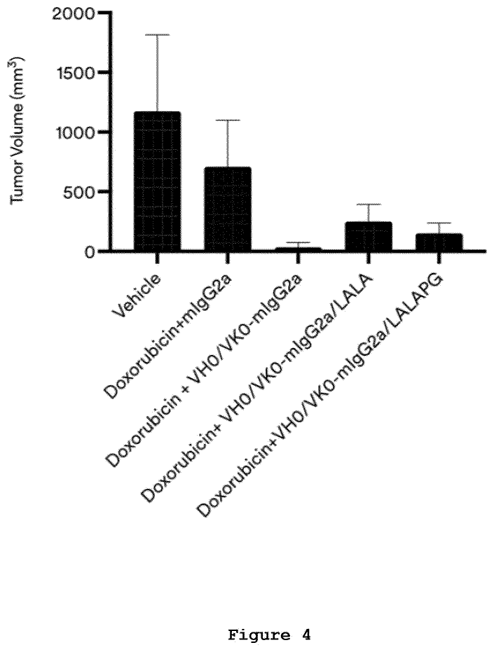

### EXAMPLES

### Immunization of Mice for Generation of Parental Monoclonal Antibody (VH0/VK0-mIgG1)

Mice were subcutaneously immunized with a 100 microgram dose of His-tagged anti-hPDL2 antigen (Sino Biologicals, cat #10292-H0H) together the Freund's adjuvant in a ratio of 1:1, every two weeks over 8 weeks for a total of 4 doses. 96 hours before animals were sacrificed for the fusion procedure, they were immunized on final time with 80 micrograms of antigen. Isolated splenocytes from sacrificed mice were pooled between two mice and fused with the P3X63 myeloma cell line and seeded in 96 well plates in HAT selection. After 10 days, supernatants of the resulting hybridomas were screened for cross-reactivity to recombinant hPD-L2 and blocking activity against the hPD-L2/hPD-1 interaction and counter-screened for cross-reactivity to the His tag by ELISA. Leads were selected from this initial screen, scaled up, purified and further confirmed for binding to hPD-L2 and blocking of the hPD-L2/hPD-1 interaction over a range of antibody dilutions and VH0/VK0-mIgG1 was selected as a lead for humanization.

### Design of Humanized Variants of VH0/VK0-mIgG1

Several residues in the variable regions of VH0/VK0-mIgG1 were first identified as being important for the binding properties of the antibody by structural modeling in Swiss PBD. Based on this, human sequences segments were selected based on non-binding to MHC class II alleles by in silico analysis. Additional, independent in silico analysis revealed developability liabilities in the sequences which were modified to enhance future process development.

Based on the composite in silico analyses, 8 variant heavy chains (VH1-8) and 3 variant light chains (VK1-3), in addition to the parental murine variant heavy and light chains (VH0/VK0), were cloned into a mammalian expression vector on the hIgG1 backbone, transiently expressed in CHO cells and purified. Assessment of these chimeric anti-PDL2 lead mAb VH0/VK0; (SEQ ID NO: 1/SEQ ID NO: 9) and humanized variants VH0/VK1 (SEQ ID NO: 1/SEQ ID NO: 10); VH1/VK0 (SEQ ID NO: 2/SEQ ID NO: 9); VH1/VK1 (SEQ ID NO: 2/SEQ ID NO: 10); VH1/VK2 (SEQ ID NO: 2/SEQ ID NO: 11); VH1/VK3 (SEQ ID NO: 2/SEQ ID NO: 12); VH2/VK1 (SEQ ID NO: 3/SEQ ID NO: 10); VH2/VK2 (SEQ ID NO: 3/SEQ ID NO: 11); VH2/VK3 (SEQ ID NO: 3/SEQ ID NO: 12); VH3/VK1 (SEQ ID NO: 4/SEQ ID NO: 10); VH3/VK2 (SEQ ID NO: 4/SEQ ID NO: 11); VH3/VK3 (SEQ ID NO: 4/SEQ ID NO: 12); VH4/VK1 (SEQ ID NO: 5/SEQ ID NO: 10); VH4/VK2 (SEQ ID NO: 5/SEQ ID NO: 11); VH4/VK3 (SEQ ID NO: 5/SEQ ID NO: 12); VH6/VK1 (SEQ ID NO: 6/SEQ ID NO: 10); VH6/VK2 (SEQ ID NO: 6/SEQ ID NO: 11); VH6/VK3 (SEQ ID NO: 6/SEQ ID NO: 12); VH7/VK1 (SEQ ID NO: 7/SEQ ID NO: 10); VH7/VK2 (SEQ ID NO: 7/SEQ ID NO: 11); VH7/VK3 SEQ ID NO: 7/SEQ ID NO: 12); VH8/VK1 (SEQ ID NO: 8/SEQ ID NO: 10); VH8/VK2 (SEQ ID NO: 8/SEQ ID NO: 11); VH8/VK3 (SEQ ID NO: 8/SEQ ID NO: 12) were transiently scaled up on a large scale. To achieve this, they were transiently transfected into CHO cells using the MaxCyte STX® electroporation system (MaxCyte Inc., Gaithersburg, USA) with OC-400 processing assemblies. Following cell recovery, cells were diluted to 3×106 cells/mL into CD Opti-CHO medium (ThermoFisher, Loughborough, UK) containing 8 mM L-Glutamine (ThermoFisher, Loughborough, UK) and 1× Hypoxanthine-Thymidine (ThermoFisher, Loughborough, UK). Two hours after resuspension 0.5× Penicillin Streptomycin solution (ThermoFisher, Loughborough, UK) was added to the culture (5 mL/L). The culture temperature was reduced 24 hours post-transfection, from 37° C. to 32° C. and 1 mM sodium butyrate (Sigma, Dorset, UK) was added. Cultures were fed 24 hours and 7 days after transfection by adding 30% and 15% (of the culture volume) CHO CD Efficient Feed B (ThermoFisher, Loughborough, UK) respectively, and 3.3% and 1.65% Function Max Titre Enhancer (ThermoFisher, Loughborough, UK) respectively. CHO supernatants were harvested 14 days post transfection. Antibody concentrations were measured on the Octet QK 384 using Protein A biosensors (Molecular Devices, Wokingham, Berkshire, UK), using an IgG1 antibody as standard.

Following culture harvest, antibody supernatants were filtered using 0.2 μM filter systems (Corning, New York, US) to remove remaining cell debris and supplemented with 10× PBS to neutralise pH. The antibodies were then purified from supernatants using 5 mL Hitrap MabSelect PrismA columns (Cytiva, Little Chalfont, UK) previously equilibrated with 10 CV (column volume) of 1× DPBS, pH 7.2-7.4. Following the sample loading, the columns were washed with 10 CV of 1× DPBS and protein eluted with 0.1 M sodium citrate, pH 3.0. Fractions were collected, and pH adjusted with 1 M Tris-HCl, pH 9.0 followed by OD280 nm quantification. Antibody-containing fractions for each variant were pooled and buffer exchanged into 1× DPBS, pH 7.2-7.4 and filter sterilised with 0.2 μM syringe filters (Sartorius, Epsom, UK) before quantification by OD280 nm using an extinction coefficient (Ec (0.1%)) based on the predicted amino acid sequence. PrismA purified antibodies were then analysed by SDS-PAGE and by analytical SE-HPLC.

### Multi-Cycle Kinetics Biacore of VH0/VK0-mIgG1 and 5 Humanized Variant Leads

In order to assess the binding of the five lead humanized variants (VH6/VK1; VH6/VK2, VH6/VK3; VH7/VK2 and VH8/VK2) to human PD-L2 antigen, multi-cycle kinetic analysis was performed on purified material. Kinetic experiments were performed at 25° C. on a Biacore 8K running Biacore Insight Evaluation software (Cytiva, Uppsala, Sweden).

HBS-EP+ (Cytiva, Uppsala, Sweden), supplemented with 1% BSA w/v (Sigma, Dorset, UK) was used as running buffer as well as for ligand and analyte dilutions. Purified lead antibodies were diluted to 1 μg/mL in running buffer and at the start of each cycle, loaded onto all of the active flow cells of a Protein A sensor chip (Cytiva, Little Chalfont, UK). Antibodies were captured at a flow rate of 10 μl/min to give an immobilisation level (RL) of ˜120 RU. The surface was then allowed to stabilise.

Multi-cycle kinetic data was obtained using human PD-L2 antigen as the analyte injected at a flow rate of 40 μl/min to minimise any potential mass transfer effects. A seven point, two-fold dilution range from 0.37 nM to 20 nM of antigen was prepared in running buffer. For each concentration, the association phases were monitored for 240 seconds and the dissociation phase was measured for 1200 seconds. Regeneration of the sensor chip surface was conducted between cycles using a single injection of 10 mM Glycine-HCl, pH1.5. Multiple repeats of a blank and of antigen were programmed into the kinetic runin order to check the stability of both the surface and analyte over the kinetic cycles.

The signal from the reference flow cell (no IgG captured) was subtracted from that of the active flow cell for each of the channels used to correct for bulk effect and differences in non-specific binding to a reference surface. The signal from each IgG blank run (IgG captured but no antigen) was subtracted to correct for differences in surface stability. The double referenced sensorgrams were fitted with the Langmuir (1:1) binding model where the closeness of fit of the data to the model is evaluated using the Chi square value which describes the deviation between the experimental and fitted (observed and expected) curves. The fitting algorithm seeks to minimize the Chi square value.

### Assay for Binding of Antibodies to Cellular Human and Mouse PD-L2

CHO-K1 cells over-expressing either hPDL2-GFP or mPD-L2 GFP were detached, washed in phosphate buffered saline (PBS) and adjusted to a cell concentration of around 1 million cells in cytometry buffer (CB, PBS containing 1% PBS). Around 80,000 cells were then distributed into a round-bottom 96 well plate and the plate was centrifuged for 3 minutes at 400 g at 4° C. and supernatant discarded. Appropriate antibody concentrations in 50 microliter volumes were added to the wells and incubated for 30 minutes at 6-8° C. The plate was washed 3 times with PBS and relevant secondary antibodies (either AlexaFluor 647 Goat anti-Mouse IgG (H+L) (Invitrogen A21235/lot 1915807) or AlexaFluor 647 Goat anti-Rat IgG (H+L) (Invitrogen A21247/lot 1921562) were added for 30 minutes at 4° C. Cells were again washed with PBS two times, resuspended in CB and analyzed in either the Sony ID7000 Spectral Cell analyzer (Sony) or the GALLIOS Flow Cytometer (Beckman-Coulter) was used. EC₅₀ was calculated using the Sigmoidal 4PL equation of the GraphPad Prism-9 software.

Assay for Blocking Activity of Antibodies of Recombinant hPD-1 to Cellular hPD-L2

CHO-K1 cells over-expressing either hPDL2-GFP were detached, washed in phosphate buffered saline (PBS) and adjusted to a cell concentration of around 1 million cells in CB. Around 80,000 cells were then distributed into a round-bottom 96 well plate and the plate was centrifuged for 3 minutes at 400 g at 4° C. and supernatant discarded. A constant concentration of ligand recombinant hPD-1 ligand (Sino Biological 10377-H08H/lot LC13JA2207) of 15 micrograms/mL was incubated with respective concentration of antibody, added to the cells and incubated for 30 min at 6-8° C. Cells were washed 3 times in PBS and then incubated with a rabbit polyclonal anti-His (Abcam Ab1187/lot GR3369369-1) in CB and subsequently washed three times in PBS. Cells were then incubated with a labeled anti-rabbit secondary (AlexaFluor 647 Goat anti-Rabbit IgG (H+L) (Invitrogen A21244/lot 1910774) for 15 minutes at 4° C., washed two times in PBS, resuspended in CB and analyzed in the Sony ID7000 Spectral Cell analyzer (Sony).

ELISA for mPDL2

ELISA plates were coated with recombinant mPDL2 (Sino Biological, cat 50804-M08H-B, lot LC10SE0706) at a concentration of 2 micrograms/milliliter at 4° C. for 15 h. Coating was then washed two times with PBS and blocked with 0.5% bovine serum albumin (BSA)/PBS at 37° C. for one hour. Blocking was removed and appropriate antibody concentrations diluted in 0.5% BSA/PBS for 1 hour at 37° C. Plates were then washed five time in PBS and a goat anti-mouse-HRP (Jackon Immuno Research cat #115-035-071) diluted in 0.5% BSA/PBS was added and incubated for 30 minutes at 37° C. Plates were washed again five times with PBS and TMB substrate (Medicago cat #10-9405) was added for 30 minutes at 37° C. in the dark. Reaction was stopped with 1N HCl and read and optical densities read on a standard spectrophotometer plate reader at 450 nM.

Reporter Assay Blocking Activity on Cellular hPD-L2/hPD-1 Interaction

Cellular blocking activity of antibodies according to the present invention was assessed by with the Promega (Southampton, Hampshire, UK) PD-1/PD-L2 blockade bioassay (Promega, Cat No. CS187131-1), which is a bioluminescent cell-based assay that is used to measure the potency and stability of antibodies and other biologics designed to block the hPD-1/PD-L2 interaction. In brief, CHO-K1 cells overexpressing hPD-L2 were thawed and plated in a 96 well plate. The following day, appropriate concentrations of antibodies were added to the wells. Jurkat PD-1 effector cells, expressing a luminescent NFAT reporter for cell activation, were subsequently thawed and added to the wells immediately. The co-culture was incubated for 6 hours and read using the SpectraMax i3× reader (Molecular Devices, Wokingham, UK). GraphPad Prism 9.0 (GraphPad Software, La Jolla, Ca) was used for the data analysis and data was plotted using a four-parameter non-linear regression model.

### Implantable Mouse PyMT Mammary Tumor Model

1 million PyMT cells were injected in PBS into the right mammary fat pad number 4, with an N=10 animals per treatment group. When tumors reached an average of 100 millimeter cubed on day 17 post-injection, treatment regimens were initiated. All mice were sorted into study groups based on caliper estimation of tumor burden. The mice were distributed to ensure that the mean tumor burden for all groups was within 10% of the overall mean tumor burden for the study population. The first dose of doxorubicin was administered intraperitoneally at 4 mg/kg, as well as the antibodies, all at doses of 10 mg/kg intraperitoneally, on day 17 post-implantation. Doxorubicin (MWI Animal Health, BJ0080A/NA) was administered once more on day 24 and antibodies on days 20, 24, 27, 31, 34, 38, 41. The following commercial antibodies were used in the study: anti-mouse PD-L2 (clone TY-25, Bio X Cell lot/cat 0411901. Vehicle-treated animals received only vehicle solvent for doxorubicin as a no-treatment control. Tumor volume was measured at least 2-3 times per week throughout the study with a standard caliper.

### Spontaneous Mouse PyMT Breast Tumor Model

Hemizygous transgenic PyMT mice were allowed to develop tumors to an average size of around 200 cubic millimeters, usually around 9 weeks, at which time treatment was initiated, with N=3-5 per treatment group. Doxorubicin (Aurovitas) was administered intraperitoneally once per week at 4 mg/kg and antibodies were administered every three days at a dose of 10 mg/kg during four weeks or 3 mg/kg where noted. Vehicle-treated animals received only vehicle solvent for doxorubicin as a no-treatment control. Tumors were measured with a standard caliper once per week. At the end of the study, tumors were resected and further analyzed by immunohistochemistry.

For the depleting study, the same treatment regimen described above was carried out but in combination with CD4 or CD8-depleting antibodies (BioXCell), administered on day −1, day 0 and weekly thereafter at at a dose of 200 micrograms per animal.

### Immunostaining of PyMT Tumors

For double SA-beta-galactosidase/PD-L2 staining, following resection, tumors were fixed in 2% paraformaldehyde and 0.2% glutaldehyde in PBS for 2 hours at room temperature. Samples were washed in PBS for 15 minutes then stained by standard procedures for SA-beta-galactosidase activity overnight. Following this, tumors were washed in PBS for 15 minutes, dehydrated in 70% ethanol and embedded in paraffin, sectioned, and stained for PDL2 with an anti-PDL2 antibody (Cell Signaling, D6L5A cat #49189) by standard immunohistochemistry techniques. To quantify the number of cells double positive for SA-beta-galactosidase and PDL2, 3 random fields from 2 mammary glands from each animal in each treatment group was analyzed with Image J software. Staining for CD3, CD4 and CD8 was also carried out by standard immunohistochemistry techniques on paraffin-embedded material and quantified according to pixel density using Image J software.

1. An isolated monoclonal antibody that specifically binds to programmed cell death ligand 2 (PD-L2) and blocks activity against PD-L2/hPD1 interaction, wherein the antibody comprises six CDR sequences in the heavy chain variable region and in the light chain variable region, and comprises
a) as heavy chain variable region CDRs
a CDRH1 region of SEQ ID NO:13 (GYAFSNYFIE) or SEQ ID NO:14 (GYSFSNYFIE);
a CDRH2 region of SEQ ID NO:15 (LNIPGSGGSNYAEKFKG); and
a CDRH3 region of SEQ ID NO: 16 (RRLPPDWYFDV); and
b) as light chain region CDRs
a CDRL1 region of SEQ ID NO: 17 (RSSQSLVHSGGNTYLH) or SEQ ID NO: 20 (RSSQSLVHSDGNTYLH);
a CDRL2 region of SEQ ID NO: 18 (KVSNRFS); and
a CDRL3 region of SEQ ID NO: 19 (SQSTHVPWT).

2. The isolated monoclonal antibody of claim 1, wherein the antibody comprises a heavy chain variable region and a light chain variable region, and comprises
a) as heavy chain variable region CDRs
b) a CDRH1 region of SEQ ID NO:14 (GYSFSNYFIE);
a CDRH2 region of SEQ ID NO:15 (LNIPGSGGSNYAEKFKG); and
a CDRH3 region of SEQ ID NO: 16 (RRLPPDWYFDV); and
c) as light chain region CDRs
a CDRL1 region of SEQ ID NO: 17 (RSSQSLVHSGGNTYLH);
a CDRL2 region of SEQ ID NO: 18 (KVSNRFS); and
a CDRL3 region of SEQ ID NO: 19 (SQSTHVPWT).

3. The isolated monoclonal antibody of claim 1, wherein the antibody comprises a heavy chain variable region and a light chain variable region, and comprises
a) as heavy chain variable region CDRs
a CDRH1 region of SEQ ID NO:13 (GYAFSNYFIE);
a CDRH2 region of SEQ ID NO:15 (LNIPGSGGSNYAEKFKG); and
a CDRH3 region of SEQ ID NO: 16 (RRLPPDWYFDV); and
b) as light chain region CDRs
a CDRL1 region of SEQ ID NO: 20 (RSSQSLVHSDGNTYLH);
a CDRL2 region of SEQ ID NO: 18 (KVSNRFS); and
a CDRL3 region of SEQ ID NO: 19 (SQSTHVPWT).

4. The isolated monoclonal antibody according to claim 1, wherein
(a) the heavy chain variable region comprises the amino acid sequence of SEQ ID NO: 1 (VH0) and the light chain variable region comprises the amino acid sequence of SEQ ID NO:9 (VK0); or
(b) the heavy chain variable region comprises the amino acid sequence of SEQ ID NO: 6 (VH6) and the light chain variable region comprises the amino acid sequence of SEQ ID NO: 12 (VK3).

5. The isolated antibody of claim 1, wherein the antibody comprises at least one of the following characteristics:
(a) the antibody binds to human PD-L2 with a dissociation constant (K_{D}) equal to or less than 10⁻¹⁰ M;
(b) the antibody blocks in vitro the interaction between human programmed cell death protein 1 (hPD-1) and human PD-L2 with an EC₅₀ equal to or less than 1.3 nM in a bioluminescent cell-based assay using Jurkat T cells expressing human PD-1 and a luciferase reporter driven by an NFAT response element (NFAT-RE) and CHO-K1 cells expressing human PD-L2 and an engineered cell surface protein designed to activate cognate TCRs in an antigen-independent manner;
(c) the antibody specifically binds both to human and murine PD-L2.

6. The isolated antibody of claim 1, wherein the antibody is a chimeric, humanized or human antibody.

7. A composition comprising the antibody of claim 1 and a pharmaceutically acceptable excipient.

8. An isolated nucleic acid sequence that encodes the amino acid sequence of the light chain variable region and/or the heavy chain variable region of the antibody of claim 1.

9. A recombinant expression vector comprising the nucleic acid of claim 8.

10. An isolated host cell comprising the recombinant expression vector of claim 9.

11. A method of treating, ameliorating or preventing a disease or condition associated to the presence of senescent cells in cancer tissue, comprising administering the antibody according to claim 1.

12. The method according to claim 11, wherein the antibody is administered in combination with a chemotherapeutic agent.

13. The method according to claim 12, wherein the chemotherapeutic agent is selected from the group consisting of alkylating agents, nitrosourea agents, antimetabolites, antitumor antibiotics, alkaloids derived from plant, topoisomerase inhibitors, hormone therapy medicines, hormone antagonists, aromatase inhibitors, P-glycoprotein inhibitors, platinum complex derivatives, growth inhibitory agents, radioactive isotopes, target agents, and immunotherapeutic agents and combinations thereof.

14. The method according to claim 12, wherein the chemotherapeutic agent is selected from the group consisting of doxorubicin, carboplatin and paclitaxel.

15. The method according to claim 11, wherein a target agent is an CDK4/6 inhibitor.

16. The method according to claim 11, wherein the disease is cancer.

17. The method according to claim 16, wherein the cancer is selected from the group consisting of breast cancer, non-small cell lung cancer, ovarian cancer, head squamous cell carcinoma, neck squamous cell carcinoma, carcinoma, squamous carcinoma in cervical canal, eyelid, tunica conjunctiva, vagina, lung, oral cavity, skin, urinary bladder, tongue, larynx or gullet, adenocarcinoma in prostate, adenocarcinoma in small intestine, adenocarcinoma in endometrium, adenocarcinoma in cervical canal, adenocarcinoma in large intestine, adenocarcinoma in lung, adenocarcinoma in pancreas, adenocarcinoma in gullet, adenocarcinoma in intestinum rectum, adenocarcinoma in uterus, adenocarcinoma in stomach, adenocarcinoma in mammary gland, adenocarcinoma in ovary, sarcomata in myogenic sarcoma, leukosis, neuroma, melanoma and lymphoma.
